(19) United States
(12) Patent Application Publication
(10) Pub. No.: US 2025/0032750 A1
(43) Pub. Date: Jan. 30, 2025

(54) CATHETER CONTROL USING SHAPE DETECTION

(71) Applicant: Covidien LP, Mansfield, MA (US)

(72) Inventors: Dany Junio, Brookline, MA (US); Paul M. Loschak, Somerville, MA (US); Matthew C. Blanco, Boston, MA (US); John M. Staton, Waltham, MA (US)

(73) Assignee: Covidien LP, Mansfield, MA (US)

(21) Appl. No.: 18/744,270

(22) Filed: Jun. 14, 2024

Related U.S. Application Data
(60) Provisional application No. 63/529,607, filed on Jul. 28, 2023.

(51) Int. Cl. A61M 25/01; A61B 1/267

(52) U.S. Cl. CPC A61M 25/0113; A61M 25/0147; A61B 1/2676; A61M 2025/0166; A61M 2205/3317; A61M 2205/3365

(57) ABSTRACT

A system and method of adjusting control parameters for a catheter including receiving sensor data from a sensor on a distal portion of a catheter, detecting a location of the distal portion of the catheter within a luminal network of a patient, determining a shape of the catheter within the luminal network of the patient, modifying control parameters of a drive motor operably coupled to the catheter based on the detected location of the distal portion of the catheter and the determined shape of the catheter within the luminal network of the patient.

### TECHNICAL FIELD

This disclosure relates to the field of navigation of and control of medical devices, such as catheters within luminal structures.

### DESCRIPTION OF RELATED ART

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient, and particularly so for areas within luminal networks of the body such as the lungs, blood vessels, colorectal cavities, and the renal ducts. To enable the endoscopic approach, navigation systems have been developed that use previously acquired MRI data or CT image data to generate a three-dimensional (3D) rendering, model, or volume of the particular body part.

The resulting volume generated from the MRI scan or CT scan may be utilized to create a navigation plan to facilitate the advancement of a navigation catheter (or other suitable medical device) through the luminal network to an area of interest. These MRI or CT scans are typically acquired at some point prior to any navigation of the patient.

During the navigation, a locating or tracking system, such as an electromagnetic (EM) tracking system or shape sensing tracking system, may be utilized in conjunction with, for example, CT data, to facilitate guidance of the navigation catheter to the area of interest. While these systems are effective, improvements are always desired.

### SUMMARY

One aspect of the disclosure is directed to a method including receiving electromagnetic (EM) sensor data from a sensor on a distal portion of a catheter. The method also includes detecting a location of the distal portion of the catheter within a luminal network of a patient. The method also includes determining a shape of the catheter within the luminal network of the patient. The method also includes modifying control parameters of a drive motor operably coupled to the catheter based on the detected location of the distal portion of the catheter and the determined shape of the catheter within the luminal network of the patient. The method also includes articulating the catheter. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method further includes comparing the determined shape of the catheter to a pathway plan; and modifying the determined shape of the catheter based on the comparison.

The drive motor is operably connected to at least one pull wire and the modification of the control parameters adjusts one or more of distance of pull of the at least one pull wire, a speed at which the at least one pull wire is driven by the drive motor, or a force applied to the at least one pull wire by the drive motor to articulate the catheter. The drive motor is located in a handle connected to the catheter, the handle including one or more input devices for controlling the drive motor. The drive motor is operably connected to a robotic arm configured to advance the catheter within the luminal network of the patient. Determination of the shape of the catheter determines the location and angularity of at least one bend of the catheter. The detecting a position of a distal portion of the catheter, determination of the shape of the catheter, and modifying control parameters of the drive motor is repeated until a distal portion of the catheter arrives proximate a target. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A second aspect of the disclosure is directed to a navigation system including a catheter having a sensor formed on a distal portion thereof; at least one pull wire integrated into and extending along a length of the catheter. The system also includes a drive motor formed on a proximal portion of the catheter, the motor operably connected to the pull wire via a threaded shaft. The system also includes a memory storing thereon an application that when executed by a processor performs steps of: receiving sensor data from the sensor, detecting a location of the distal portion of the catheter within a luminal network of a patient, determining a shape of the catheter within the luminal network of the patient, modifying control parameters of the drive motor based on the detected location of the distal portion of the catheter and the determined shape of the catheter within the luminal network of the patient, and driving the drive motor to articulate the distal portion of the catheter. Other embodiments of this aspect include corresponding computer systems, apparatus, and computer programs recorded on one or more computer storage devices, each configured to perform the actions of the methods and systems described herein.

Implementation of this aspect of the disclosure may include one or more of the following features. The system where the determined shape of the catheter is based on the received sensor data from the sensor. The application, when executed by the processor compares the determined shape of the catheter to a pathway plan of the luminal network; and modifies the determined shape of the catheter based on the comparison. Modifying the control parameter adjusts one or more of distance of pull of the at least one pull wire, a speed at which the at least one pull-wire is driven by the drive motor, or a force applied to the at least one pull wire by the drive motor to articulate the catheter. The drive motor is located in a handle connected to the catheter, the handle including one or more input devices for controlling the drive motor. The drive motor is operably connected to a robotic arm configured to advance the catheter within the luminal network of the patient. Determining the shape of the catheter determines the location and angularity of at least one bend of the catheter. The detecting a position of a distal portion of the catheter, determination of the shape of the catheter, and modifying control parameters of the drive motor is repeated until a distal portion of the catheter arrives proximate a target. The sensor is one or more of a fiber, an electromagnetic (EM) sensor, an inertial measurement unit (IMU), or an ultrasonic sensor. The system further includes a second pull wire integrated into and extends the length of the catheter, the second pull wire operably connected to the drive motor and configured to articulate the catheter in a direction opposite the at least one pull wire. The second pull wires are integrated into and extend the length of the catheter and actuation of the second motor articulates the catheter in directions substantially orthogonal to a direction of articulation caused by the at least one pull wire and second pull wire. The system further includes a third drive motor operably connected to the catheter and configured to rotate the catheter about its longitudinal axis. The sensor is an electromagnetic sensor configured to detect electromagnetic fields generated by a transmitter mat. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and of the disclosure are described hereinbelow with references to the drawings, wherein:

FIG. 1 is a schematic view of a luminal network navigation system in accordance with the disclosure;

FIG. 2 is a flow chart of a method of controlling a catheter in accordance with the disclosure;

FIGS. 3A and 3B depict aspects of an articulation system for a catheter in accordance with the disclosure;

FIG. 4 is a 3D model generated as part of a pathway plan in accordance with the disclosure;

FIG. 5 is a schematic view of an imaging and computing system in accordance with the disclosure.

### DETAILED DESCRIPTION

Catheters and catheter like devices, such as endoscopes, are used in a myriad of medical procedures. These flexible devices are typically used to navigate through luminal networks of the body including the vasculature, airways, and digestive systems. In accordance with the disclosure, to aide in navigating to a specific location, the distal tip of the catheter can be articulated, deflected, or rotated by a user through controls on the catheter proximal end or via a robotic interface outside the body. These manipulations allow the tip to point towards and enter branching structures. Upon arrival at the desired anatomic location a medical procedure may be performed.

A common unmet need across all of them is that it would take significant efforts for a physician to develop proficiency in manipulating these tools. The main challenge rises from the fundamental limitation of the human brain to process high-dimensional data. Specifically, the physician often needs to mentally register the position and orientation of a 3D object (i.e., catheter) onto a 2D display (e.g., images in endoscopy, fluoroscopy, ultrasonography, etc.) while concurrently deciding how to turn handle knobs to steer the catheter towards the desired direction. Further, the absence of palpable feedback to the clinician makes it challenging to effectively control the motor driven catheter. On the one hand, this cognitive burden increases the risk of operator-related medical errors. On the other hand, the demanding eye-hand coordination restricts the product to be used by well-trained elite physicians only, preventing it from reaching other less privileged customers.

This disclosure describes systems and methods to significantly enhance the automatic navigation and catheter control during navigation procedures. Aspects of the disclosure are directed to semi-autonomous and/or autonomous (e.g., robotic) navigation of a catheter to the most distal regions of the lungs, with both accuracy and repeatability. These methods can be employed in a variety of products used in different minimally invasive procedures including but not limited to bronchoscopy.

One aspect of the disclosure is directed to the control applied to the catheter as it navigates different portions of the airways of the lungs. During navigation of the central airways, there are competing challenges of little to no resistance to movement of the catheter but a greater magnitude of articulation of the distal portion of the catheter may be needed to allow advancement into an airway whose opening is at a large angle relative to airway in which the catheter is already located. As navigation proceeds towards the periphery the airways become smaller and begin to impact the navigation of the catheter. Not only do the airways create friction as a result of the catheter-to-tissue contact, but the airways themselves can move as a result of forces applied by the catheter to the tissue. Further, as the catheter moves further into the periphery smaller and more controlled movements may be more appropriate to enable efficient navigation into smaller branching airways. Still further, as the shape of the catheter incorporates more and more bends, the shape of the catheter impacts how forces applied via a motor at a proximal end are carried through the catheter to effectuate articulation at the distal end.

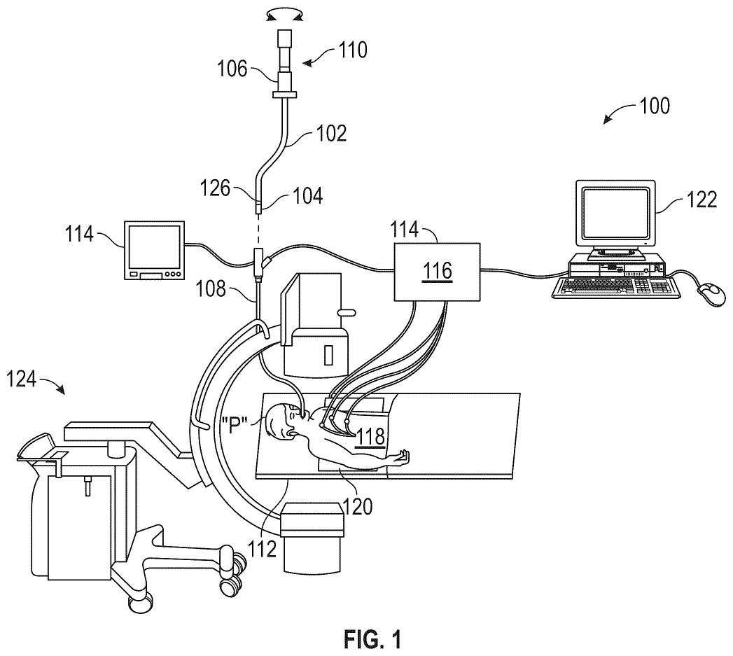

FIG. 1 is a perspective view of an exemplary system for facilitating navigation of a medical device, e.g., a catheter to a soft tissue target via airways of the lungs. As shown in FIG. 1, catheter 102 is part of a catheter guide assembly 106. In one aspect, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. The catheter 102 may itself include imaging capabilities via an integrated camera or optics component 109 and a separate bronchoscope 108 is not strictly required. A locatable guide (LG) 110 (a second catheter), including a sensor 104 may be inserted into catheter 102 and locked into position such that sensor 104 extends a desired distance beyond the distal tip of catheter 102. The position and orientation of sensor 104 relative to a reference coordinate system, and thus the distal portion of catheter 102, within an electromagnetic field can be derived. Catheter guide assemblies 106 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE™ Procedure Kits, and are contemplated as useable with the disclosure.

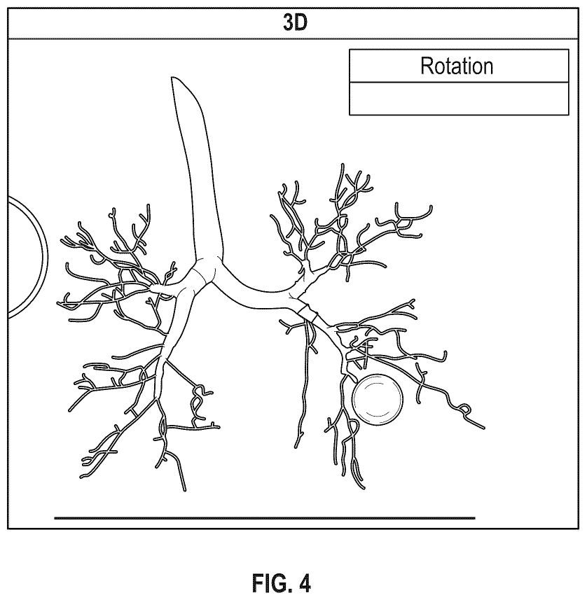

System 100 generally includes an operating table 112 configured to support a patient P and monitoring equipment 114 coupled to bronchoscope 108 or catheter 102 (e.g., a video display, for displaying the video images received from the video imaging system of bronchoscope 108 or the catheter 102); a locating or tracking system 114 including a locating module 116, a plurality of reference sensors 18 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 102, or a suitable device therethrough, relative to the target. FIG. 4 depicts a 3D model generated a part of a pre-procedure pathway plan, used to guide the clinician as they navigate a catheter 102 to a target (shown in green). The 3D model may be employed in the methods described herein below.

As is typical of catheter guide assembly 10 navigation the six degrees-of-freedom electromagnetic locating or tracking system 114, or other suitable system for determining position and orientation of a distal portion of the catheter 102, is utilized, as will be outlined below for performing registration of a detected position of the sensor 104 and a 3D model generated from a CT or MRI image scan. Tracking system 114 includes the tracking module 116, a plurality of reference sensors 118, and the transmitter mat 120 (including the markers). Tracking system 114 is configured for use with a locatable guide 110 and particularly sensor 104. As described above, locatable guide 110 and sensor 104 are configured for insertion through catheter 102 into patient P's airways (either with or without bronchoscope 108) and are selectively lockable relative to one another via a locking mechanism.

Transmitter mat 120 is positioned beneath patient P. Transmitter mat 120 generates an electromagnetic field around at least a portion of the patient P within which the position of a plurality of reference sensors 118 and the sensor 104 can be determined with use of a tracking module 116. In some embodiments, a second electromagnetic sensor 126 may also be incorporated into the end of the catheter 102. The second electromagnetic sensor 126 may be a five degree-of-freedom sensor or a six degree-of-freedom sensor and may be employed in addition to or as an alternative to the sensor 104 in the LG 110. One or more of reference sensors 118 are attached to the chest of the patient P. Registration is generally performed to coordinate locations of the three-dimensional model and two-dimensional images from the planning phase, with the patient P's airways as observed through the bronchoscope 108 and allow for the navigation phase to be undertaken with knowledge of the location of the sensor 104.

Registration of the patient P's location on the transmitter mat 120 may be performed by moving sensor 104 or 126 through the airways of the patient P. More specifically, data pertaining to locations of sensor 104 or 126, while the catheter 102 and/or locatable guide 110 is moving through the airways, is recorded using transmitter mat 120, reference sensors 118, and tracking system 114. A shape resulting from this location data is compared to an interior geometry of passages of a 3D model (e.g., the 3D model of FIG. 4), and a location correlation between the shape and the 3D model based on the comparison is determined, e.g., utilizing the software on computing device 122. In addition, the software identifies non-tissue space (e.g., air filled cavities) in the 3D model. The software aligns, or registers, an image representing a location of sensor 104 or 126 with the three-dimensional model and/or two-dimensional images generated from the three-dimension model, which are based on the recorded location data and an assumption that locatable guide 110 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

Though described herein with respect to EMN systems using EM sensors, the instant disclosure is not so limited and may be used in conjunction with flexible sensors such as fiber-bragg grating sensors, inertial measurement unit (IMU), ultrasonic sensors, or without sensors. Additionally, as outlined below the methods described herein may be used in conjunction with robotic systems such that robotic actuators drive the catheter 102 or bronchoscope 108 proximate the target.

In accordance with aspects of the disclosure, the visualization of intra-body navigation of a medical device (e.g., a biopsy tool or a therapy tool), towards a target (e.g., a lesion) may be a portion of a larger workflow of a navigation system. An imaging device 124 (e.g., a CT imaging device such as Medtronic plc's O-arm™ system or other cone-beam computed tomography (CBCT) device) capable of acquiring 2D and 3D images or video of the patient P is also included in this particular aspect of system 100. The images, sequence of images, or video captured by imaging device 124 may be stored within the imaging device 124 or transmitted to computing device 122 for storage, processing, and display. Additionally, imaging device 124 may move relative to the patient P so that images may be acquired from different angles or perspectives relative to patient P to create a sequence of images, such as a fluoroscopic video. The pose of imaging device 124 relative to patient P while capturing the images may be estimated via markers incorporated with the transmitter mat 120. The markers are positioned under patient P, between patient P and operating table 112 and between patient P and a radiation source or a sensing unit of imaging device 124. The markers incorporated with the transmitter mat 120 may be two separate elements which may be coupled in a fixed manner or alternatively may be manufactured as a single unit. Imaging device 124 may include a single imaging device or more than one imaging device.

Computing device 122 may be any suitable computing device including a processor and storage medium, wherein the processor is capable of executing instructions stored on the storage medium. Computing device 122 may further include a database configured to store patient data, CT data sets including CT images, fluoroscopic data sets including images and video, 3D reconstruction, navigation plans, and any other such data. Although not explicitly illustrated, computing device 122 may include inputs, or may otherwise be configured to receive, CT data sets, fluoroscopic images/video and other data described herein. Additionally, computing device 122 includes a display configured to display graphical user interfaces. Computing device 122 may be connected to one or more networks through which one or more databases may be accessed.

The catheter guide assembly 106, may be manually operated for manipulation of the distal portion of the catheter 102. Additionally or alternatively, in accordance with the disclosure pull wires, described herein below, enable manipulation and shaping of a distal portion of the catheter 102. The pull wires may be connected to a motor drive mechanism. That motor drive mechanism may be handheld and include one or more buttons or joysticks to enable manipulation of the distal portion of the catheter 102. Alternatively, the catheter guide assembly 106 and any motor drive mechanism may be configured for integration onto a robotic assembly and articulation and control of the pull wires may be undertaken via computing device 122 and associated input devices (e.g., a hand-held controller similar to a game controller).

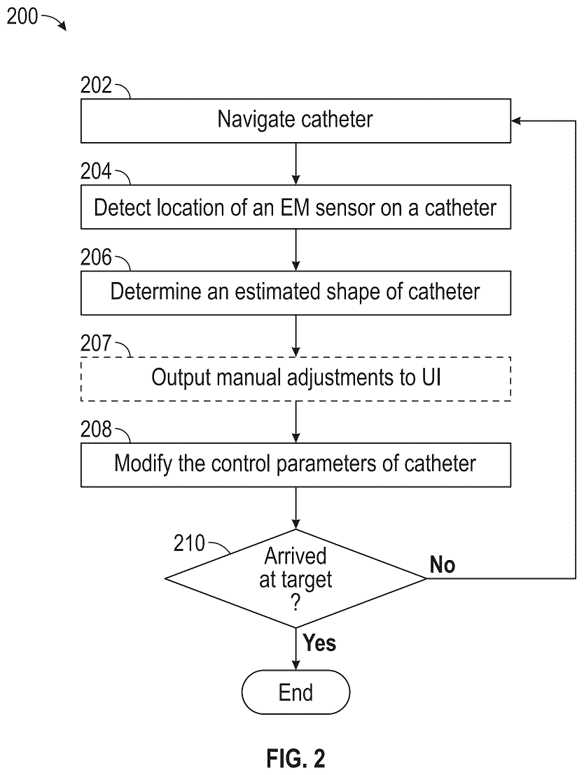

FIG. 2 depicts a method 200 in accordance with the disclosure. As noted above, the catheter 102 is configured for navigation within the lungs of a patient. At step 202 navigation of the catheter 102 commenced. A sensor 104, 126 on a distal portion of the catheter 102 detects electromagnetic fields generated by the transmitter mat 120. As with during the registration process, described above, the location of the sensor 104, 126 is detected at step 204. The detection of the location of the sensor 104, 126 occurs continuously during the navigation at a set period (e.g., 20 times per second). The data from the detected location of the sensor 104 is collected by an application running on the computing device 122. The application, using the data of the sensor position 104 collected during the navigation is assembled to form an estimated shape of the catheter 102 at any point during the navigation at step 206. The catheter 102 generally stays within the airways of the lungs of the patient during navigation, and the estimated shape of the catheter can be compared to the shape of the airways along the pathway plan from the pre-procedure planning to the current location of the sensor 104. This comparison can be used to further refine the estimated shape of the catheter 102. With the shape of the catheter 102 estimated, the control parameters of, for example, a motor drive device (described below) can be modified at step 208. For example, and as noted above, during the central navigation faster and more extreme (larger angle) manipulations of the pull wires may be required to change the shape of the distal portion of the catheter 102 and allow for entry of airways. Similarly, as the catheter 102 approaches the periphery, smaller and less rapid manipulations of the pull wires may be employed. As will be appreciated, at the periphery of the lungs the tissue is very flexible and the manipulation of the catheter 102 via the pull wires can distort the shape of the lungs. Large or forceful movements of the catheter 102 can cause the tissue of the lungs to move with the catheter 102, this movement of the lungs can lead to greater instances of CT-to-body divergence. As the tissue is distorted the tissue is further changed as compared to its positioning during the capture of the pre-procedure imaging (e.g., CT or MRI). Accordingly, as a result of the modification of the control parameters 208, the force applied by the catheter 102 can be adjusted as appropriate for the location within the lungs.

As an alternative, where rather than motorized (e.g., semi-autonomous or autonomous robotic) movement of the catheter 102 via a motor drive device, manual manipulations of the catheter 102 are undertaken, the estimated shape of the catheter at step 206 can be used to calculate manual adjustments to catheter 102 and output these to the user at step 207. As an example, the calculated manual adjustments may be displayed on a user interface of the computing device 122 and may be accompanied by audible and visual directions for adjusting the position and orientation of the catheter 102 using one or more manipulators on a proximal portion of the catheter 102 (e.g., a handle).

Further, as is understood, the shape of the catheter 102 changes how forces applied on a proximal end (e.g., at a handle or robotic interface) and is translated to the distal portion by the pull wires. The shape of the catheter 102, and particularly sharp bends (or high angularity bends) in the catheter 102, result in a need to change the distance a pull wire is actuated, or force applied to the pull wire, to achieve a desired change in shape of the distal portion of the catheter 102. For example, if navigation is to one of the upper lobes of the lungs, a near 180-degree bend is required in the catheter 102 as the catheter 102 descends the trachea and then is manipulated (e.g., via the pull wires) to orient the distal portion towards the bifurcation leading to the upper lobe. As will be appreciated, though the distal portion of the catheter 102 may be the only portion articulated by the pull-wire, after advancement through the bifurcation of the airway, the exemplary near 180-degree bend in the catheter 102 remains in the catheter 102 and advances proximally along the catheter 102 as the distal portion is advanced further into the airways. At each bifurcation an additional bend is formed in the catheter 102 until a determination is made at step 210 that the distal portion of the catheter 102 has arrived proximate the target at step 210. If it has the method 200 may stop, if not the method continues by returning to step 202. With each bend of the catheter 102 the distance of pull of the pull-wire and the force required to achieve that distance of pull changes. As a result, along with adjusting the control parameters based on the detected location of the sensor 104, 126, the control parameters are also adjusted at step 208 based on the determined shape of the catheter 102, taking into account the changes necessitated based on the bends of the catheter 102 (e.g., the shape of the catheter 102).

Still further, the shape of the catheter 102 can impact not just the distance of pull on a pull-wire but also the number of pull wires employed (where more than one is employed) and the distances or movement and forces applied to each of the pull wires. For example, when the catheter 102 is straight bending the catheter to the left 0.5 mm might require shortening a length of one pull wire 1 mm. Where, however, the catheter 102 has a curved shape after having been navigated through multiple bifurcations of the airways, bending the catheter 102 approximately 0.5 mm to the left might require shortening a first pull wire by 0.75 mm and shortening a second pull wire by 0.5 mm. Similarly, the forces applied by the pull wires to achieve the 0.5 mm bend to the left may be apportioned between the two pull wires. These and other factors can be included as part of the control parameters being modified at step 208.

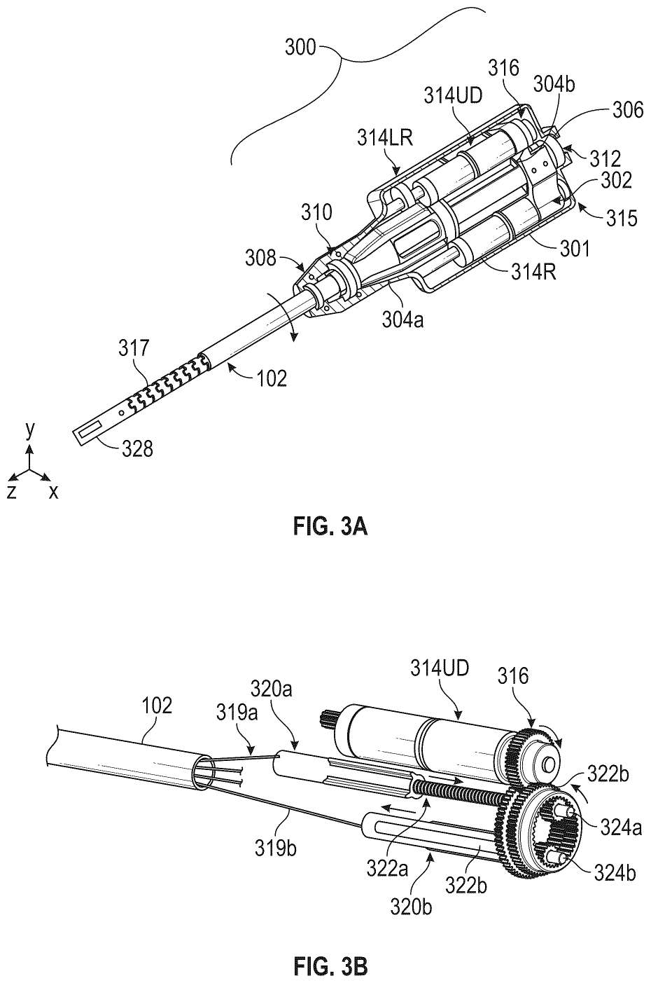

In accordance with the disclosure, the catheter 102 and its articulation and orientation relative to a target is achieved using a catheter drive mechanism 300. One example of such a drive mechanism can be seen in FIG. 3A which depicts a housing including three drive motors to manipulate a catheter extending therefrom in 5 degrees of freedom (e.g., left, right, up, down, and rotation). Other types of drive mechanisms including fewer or more degrees of freedom and other manipulation techniques may be employed without departing from the scope of the disclosure.

As noted above, FIG. 3A depicts a drive mechanism 300 housed in a body 301 and mounted on a bracket 302 which integrally connects to the body 301. The catheter 102 connects to and in one aspect of the disclosure forms an integrated unit with internal casings 304a and 304b and connects to a spur gear 306. This integrated unit is rotatable in relation to the housing 301, such that the catheter 102, internal casings 304 a-b, and spur gear 306 can rotate about shaft axis “z”. The catheter 102 and integrated internal casings 304 a-b are supported radially by bearings 308, 310, and 312. Though drive mechanism 300 is described in detail here, other drive mechanisms may be employed to enable a robot or a clinician to drive the catheter to a desired location without departing from the scope of the disclosure.

An electric motor 314R, may include an encoder for converting mechanical motion into electrical signals and providing feedback to the computing device 122. Further, the electric motor 314R (R indicates this motor if for inducing rotation of the catheter 102) may include an optional gear box for increasing or reducing the rotational speed of an attached spur gear 315 mounted on a shaft driven by the electric motor 314R. Electric motors 314LR (LR referring to left-right movement of an articulating portion 317 of the catheter 102) and 314UD (referring to up-down movement of the articulating portion 317), each motor optionally includes an encoder and a gearbox. Respective spur gears 316 and 318 drive up-down and left-right pull wires, as will be described in greater detail below. All three electric motors 314 R, LR, and UD are securely attached to the stationary frame 302, to prevent their rotation and enable the spur gears 315, 316, and 318 to be driven by the electric motors.

FIG. 3B depicts details of the mechanism causing articulating portion 317 of catheter 102 to articulate. Specifically, the following depicts the manner in which the up-down articulation is contemplated in one aspect of the disclosure. Such a system alone, coupled with the electric motor 314UD for driving the spur gear 316 would accomplish articulation as described above in a two-wire system. However, where a four-wire system is contemplated, a second system identical to that described immediately hereafter, can be employed to drive the left-right cables. Accordingly, for ease of understanding just one of the systems is described herein, with the understanding that one of skill in the art would readily understand how to employ a second such system in a four-wire system. Those of skill in the art will recognize that other mechanisms can be employed to enable the articulation of a distal portion of a catheter and other articulating catheters may be employed without departing from the scope of the disclosure.

To accomplish up-down articulation of the articulating portion 317 of the catheter 102, pull wires 319 a-b may be employed. The distal ends of the pull wires 319 a-b are attached to, or at, or near the distal end of the catheter 102. The proximal ends of the pull wires 319 a-b are attached to the distal tips of the posts 320 a, and 320 b. As shown in FIG. 3B, the posts 320a and 320b reciprocate longitudinally, and in opposing directions. Movement of the posts 320a causes one pull wire 319 a to lengthen and at the same time, opposing longitudinal movement of post 320 b causes pull wires 319 b to effectively shorten. The combined effect of the change in effective length of the pull wires 319 a-b is to cause the articulating portion 317 of catheter 102 shaft to be compressed on the side in which the pull wire 319 b is shortened, and to elongate on the side in which pull wire 319 a is lengthened.

The opposing posts 320a and 320b have internal left-handed and right-handed threads, respectively, at least at their proximal ends. As shown in FIG. 3A housed within casing 304 b are two threaded shafts 322a and 322b, one is left-hand threaded and one right-hand threaded, to correspond and mate with posts 320a and 320b. The shafts 322a and 322b have distal ends which thread into the interior of posts 320a and 320b and proximal ends with spur gears 324a and 324b. The shafts 322a and 322b have freedom to rotate about their axes. The spur gears 324a and 324b engage the internal teeth of planetary gear 326. The planetary gear 326 also has an external tooth which engages the teeth of spur gear 318 on the proximal end of electric motor 314UD.

To articulate the catheter in the upwards direction, a clinician may activate via an activation switch (not shown) for the electric motor 314UD causing it to rotate the spur gear 318, which in turn drives the planetary gear 326. The planetary gear 326 is connected through the internal gears 324a and 324b to the shafts 322a and 322b. The planetary gear 326 will cause the gears 324a and 324b to rotate in the same direction. The shafts 322a and 322b are threaded, and their rotation is transferred by mating threads formed on the inside of posts 320a and 320b into linear motion of the posts 320a and 320b. However, because the internal threads of post 320 a are opposite that of post 320b, one post will travel distally and one will travel proximally (i.e., in opposite directions) upon rotation of the planetary gear 326. Thus, the upper pull wire 319 a is pulled proximally to lift the catheter 102, while the lower pull wire 319 b must be relaxed. As stated above, this same system can be used to control left-right movement of the end effector, using the electric motor 314LR, its spur gear 316, a second planetary gear (not shown), and a second set of threaded shafts 322 and posts 320 and two more pull wires 319. Moreover, by acting in unison, a system employing four steering cables can approximate the movements of the human wrist by having the three electric motors 314 and their associated gearing and pull wires 319 computer controlled (e.g., via a handle with finger controls or a robotic interface). As will be appreciated the first pair of pull wires 319 articulate the catheter 102 in a plane substantially orthogonal a plane in which the second pair of pull wires articulate the catheter 102. Using both pairs of pull wires enable articulation in nearly any direction from the longitudinal axis of the catheter 102.

In accordance with one aspect of the disclosure, as the catheter 102 is advanced into the luminal network of a patient (e.g., the airways of the lungs), an application on the computing device, can receive inputs from the sensor 104, 126 and direct the electric motors 314 to articulate or rotate the catheter 102 to be advanced along a path to a target within the patient taking into account the modification of the control parameters (step 208) to account for the location of the sensor 104, 126 and the shape of the catheter 102.

The catheter assembly 106 may be handheld by the clinician and as the clinician advances the catheter 102 into the patient, the application makes the determination of articulations of the end of the catheter 102 required to allow the catheter 102 to reach a target location. Further, the drive mechanism 300 may be incorporated into one or more robotic arms or a sled (not shown) for movement of the catheter 102 and drive mechanism 300 in the z-direction (along the longitudinal axis of the catheter 102).

The drive mechanism 300 may receive inputs from computing device 122 or another mechanism through which the surgeon specifies the desired action of the catheter 102. Where the clinician controls the movement of the catheter 102, this control may be enabled by a directional button, a joystick such as a thumb operated joystick, a toggle, a pressure sensor, a switch, a trackball, a dial, an optical sensor, and any combination thereof. The computing device 122 responds to the user commands by sending control signals to the motors 314. The encoders of the motors 314 provide feedback to the control unit about the current status of the motors 314.

In accordance with the disclosure, and as outlined in greater detail below, the drive mechanism 300 receives signals derived by the computing device 122 to drive the catheter 102 (e.g., extend and retract pull-wires) to maintain the orientation of the distal tip of the catheter 102 despite extension of a tool such as a biopsy needle or ablation catheter or movements caused by respiration and cardiac cycles.

As described in connection with FIGS. 3A and 3B, catheter 102 is operated on its proximal end through a collection of controls for rotation and distal tip deflection. In contrast, to the aspects described in connection with FIGS. 3A and 3B, a manually advanced catheter 102 may not include the motor 314R, relying instead on manual manipulation for rotation of the catheter 102. Alternatively, the drive mechanism may include only a single pull wire 319, or a single pair of pull wires 319 a, 319 b. Articulation is thus enabled by a single or by a pair of pull wires in opposite directions. One or more knobs or levers or wheels on the proximal handle control or energize the energize the respective motor 314 to enable for distal tip articulation. Rotation and advancement/extraction of the catheter 102 are controlled directly by the user's hand pushing, pulling, and rotating the catheter 102 within the patient. As described in connection with FIGS. 3A and 3B, any or all of these manual controls can be removed, and users indirectly control the catheter operation through an interface to the motors such as a joystick that may be located on a handle (not shown) connected to the catheter 102 or connected to the computing device 122. Navigation may also be fully automatic with user oversight.

Once navigation of the catheter 102 to a position proximate target tissue (e.g., a tumor or lesion) a medical procedure may be initiated. For example, the LG 110 with sensor 104 may be removed from the catheter 102. Once removed, a biopsy tool or a therapy tool (e.g., microwave, radiofrequency, cryosurgical, or chemical ablation catheters) can be inserted through the lumen of the catheter 102 to interact with the tumor or lesion (e.g., collect biopsy or treat tumor).

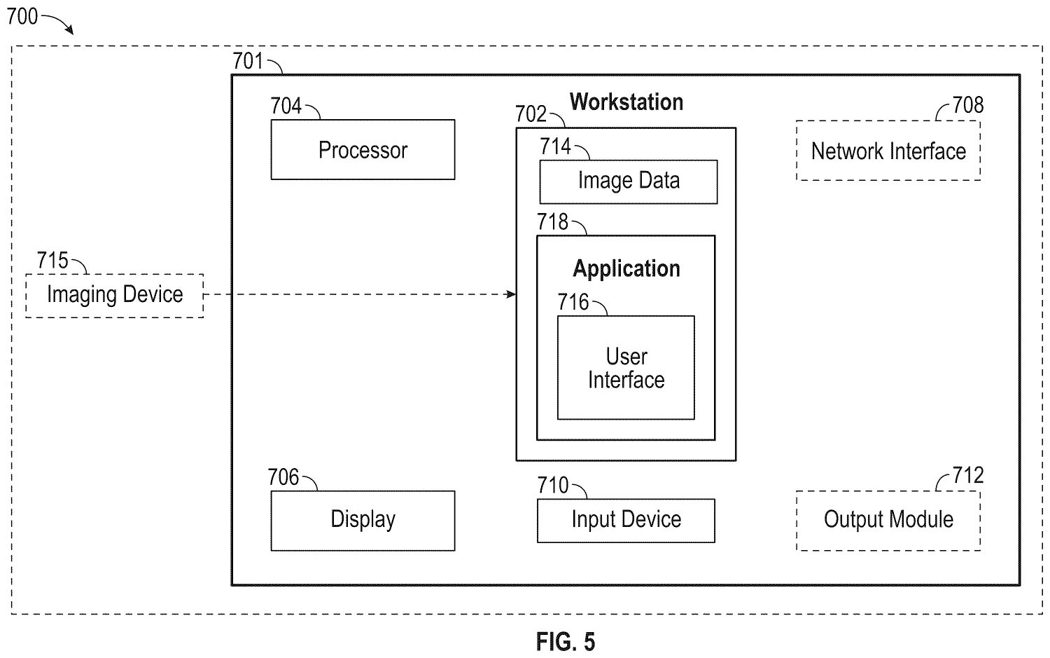

Reference is now made to FIG. 5, which is a schematic diagram of a system 700 configured for use with the methods of the disclosure including the method 200 of FIG. 2. System 700 may include a workstation 701, and optionally an imaging device 715 (e.g., a fluoroscope or an ultrasound device). Workstation 701 may be coupled with imaging device 715, directly or indirectly, e.g., by wireless communication. Workstation 701 may include a memory 702, a processor 704, a display 706 and an input device 710. Processor or hardware processor 704 may include one or more hardware processors. Workstation 701 may optionally include an output module 712 and a network interface 708. Memory 702 may store an application 718 and image data 714. Application 718 may include instructions executable by processor 704 for executing the methods of the disclosure including the method of FIG. 2.

Application 718 may further include a user interface 716. Image data 714 may include the CT scans, the generated fluoroscopic 3D reconstructions of the target area and/or any other fluoroscopic image data and/or the generated one or more slices of the 3D reconstruction. Processor 704 may be coupled with memory 702, display 706, input device 710, output module 712, network interface 708 and imaging device 715. Workstation 701 may be a stationary computing device, such as a personal computer, or a portable computing device such as a tablet computer. Workstation 701 may embed a plurality of computer devices.

Memory 702 may include any non-transitory computer-readable storage media for storing data and/or software including instructions that are executable by processor 704 and which control the operation of workstation 701 and may also control the operation of imaging device 715. Imaging device 715 may be used to capture a sequence of fluoroscopic images based on which the fluoroscopic 3D reconstruction is generated and to capture a live 2D fluoroscopic view according to this disclosure. The memory 702 may include one or more storage devices such as solid-state storage devices, e.g., flash memory chips. Alternatively, or in addition to the one or more solid-state storage devices, memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 701.

Application 718 may, when executed by processor 704, cause display 706 to present user interface 716. User interface 716 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. User interface 716 may be further configured to display the target mark in different colors depending on whether the medical device tip is aligned with the target in three dimensions.

Network interface 708 may be configured to connect to a network such as a local area network (LAN) consisting of a wired network and/or a wireless network, a wide area network (WAN), a wireless mobile network, a Bluetooth network, and/or the Internet. Network interface 708 may be used to connect between workstation 701 and imaging device 715. Network interface 708 may also be used to receive image data 714. Input device 710 may be any device by which a user may interact with workstation 701, such as, for example, a mouse, keyboard, foot pedal, touch screen, and/or voice interface. Output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

The disclosure is further described in the following numbered examples.

Example 1—A method comprising:
receiving electromagnetic (EM) sensor data from a sensor on a distal portion of a catheter;
detecting a location of the distal portion of the catheter within a luminal network of a patient;
determining a shape of the catheter within the luminal network of the patient;
modifying control parameters of a drive motor operably coupled to the catheter based on the detected location of the distal portion of the catheter and the determined shape of the catheter within the luminal network of the patient; and
articulating the catheter.

Example 2—The method of Example 1, further comprising:
comparing the determined shape of the catheter to a pathway plan; and
modifying the determined shape of the catheter based on the comparison.

Example 3—The method of examples 1 or 2, wherein the drive motor is operably connected to at least one pull wire and the modification of the control parameters adjusts one or more of a distance of pull of the at least one pull wire, a speed at which the at least one pull wire is driven by the drive motor; or a force applied to the at least one pull wire by the drive motor to articulate the catheter.

Example 4—The method of one of the preceding examples, wherein:
the drive motor is located in a handle connected to the catheter, the handle including one or more input devices for controlling the drive motor; and/or
the drive motor is operably connected to a robotic arm configured to advance the catheter within the luminal network of the patient.

Example 5—The method of claim one of the preceding examples wherein determination of the shape of the catheter determines a location and angularity of at least one bend of the catheter; and/or
detecting a location of a distal portion of the catheter, determination of the shape of the catheter, and modifying control parameters of the drive motor is repeated until the distal portion of the catheter arrives proximate a target.

Example 6-A navigation system comprising:
a catheter including a sensor formed on a distal portion thereof;
at least one pull wire integrated into and extending along a length of the catheter;
a drive motor formed on a proximal portion of the catheter, the motor operably connected to the pull wire via a threaded shaft;
a memory storing thereon an application that when executed by a processor performs steps of:
   receiving sensor data from the sensor;
   detecting a location of the distal portion of the catheter within a luminal network of a patient;
   determining a shape of the catheter within the luminal network of the patient;
   modifying control parameters of the drive motor based on the detected location of the distal portion of the catheter and the determined shape of the catheter within the luminal network of the patient; and
   driving the drive motor to articulate the distal portion of the catheter.

Example 7—The system of Example 6, wherein the determined shape of the catheter is based on the received sensor data from the sensor; and/or the application when executed by the processor:
compares the determined shape of the catheter to a pathway plan of the luminal network; and modifies the determined shape of the catheter based on the comparison.

Example 8—The system of examples 6 or 7, wherein modifying the control parameter adjusts one or more of a distance of pull of the at least one pull wire, a speed at which the at least one pull-wire is driven by the drive motor, or a force applied to the at least one pull wire by the drive motor to articulate the catheter.

Example 9—The system of examples 6-8, wherein the drive motor is located in a handle connected to the catheter, the handle including one or more input devices for controlling the drive motor or
the drive motor is operably connected to a robotic arm configured to advance the catheter within the luminal network of the patient.

Example 10—The system of examples 6-9, wherein determining the shape of the catheter determines a location and angularity of at least one bend of the catheter; and/or
detecting a location of a distal portion of the catheter, determination of the shape of the catheter, and modifying control parameters of the drive motor is repeated until a distal portion of the catheter arrives proximate a target.

Example 11—The system of examples 6-10, wherein the sensor is one or more of a fiber, an electromagnetic (EM) sensor, an inertial measurement unit (IMU), or an ultrasonic sensor.

Example 12—The system of examples 6-11, further comprising a second pull wire integrated into and extending the length of the catheter, the second pull wire operably connected to the drive motor and configured to articulate the catheter in a direction opposite the at least one pull wire.

Example 13—The system of example 12, further comprising a second drive motor operably connected to a second pair of pull wires, wherein the second pair of pull wires are integrated into and extend the length of the catheter and actuation of the second motor articulates the catheter in directions substantially orthogonal to a direction of articulation caused by the at least one pull wire and the second pull wire.

Example 14—The system of example 13, further comprising a third drive motor operably connected to the catheter and configured to rotate the catheter about its longitudinal axis.

Example 15—The system of examples 6-14, wherein the sensor is an electromagnetic sensor configured to detect electromagnetic fields generated by a transmitter mat.

While detailed aspects are disclosed herein, they are merely examples of the disclosure, which may be embodied in various forms and aspects. Therefore, specific structural and functional details disclosed herein are not to be interpreted as limiting, but merely as a basis for the claims and as a representative basis for teaching one skilled in the art to variously employ the disclosure in virtually any appropriately detailed structure.

We claim:

1. A method comprising:
receiving electromagnetic (EM) sensor data from a sensor on a distal portion of a catheter;
detecting a location of the distal portion of the catheter within a luminal network of a patient;
determining a shape of the catheter within the luminal network of the patient;
modifying control parameters of a drive motor operably coupled to the catheter based on the detected location of the distal portion of the catheter and the determined shape of the catheter within the luminal network of the patient; and
articulating the catheter.

2. The method of claim 1, further comprising:
comparing the determined shape of the catheter to a pathway plan; and
modifying the determined shape of the catheter based on the comparison.

3. The method of claim 1, wherein the drive motor is operably connected to at least one pull wire and the modification of the control parameters adjusts one or more of a distance of pull of the at least one pull wire, a speed at which the at least one pull wire is driven by the drive motor, or a force applied to the at least one pull wire by the drive motor to articulate the catheter.

4. The method of claim 1, wherein the drive motor is located in a handle connected to the catheter, the handle including one or more input devices for controlling the drive motor.

5. The method of claim 1, wherein the drive motor is operably connected to a robotic arm configured to advance the catheter within the luminal network of the patient.

6. The method of claim 1, wherein determination of the shape of the catheter determines a location and angularity of at least one bend of the catheter.

7. The method of claim 1, wherein the detecting a location of a distal portion of the catheter, determination of the shape of the catheter, and modifying control parameters of the drive motor is repeated until the distal portion of the catheter arrives proximate a target.

8. A navigation system comprising:
a catheter including a sensor formed on a distal portion thereof;
at least one pull wire integrated into and extending along a length of the catheter;
a drive motor formed on a proximal portion of the catheter, the motor operably connected to the pull wire via a threaded shaft;
a memory storing thereon an application that when executed by a processor performs steps of:
receiving sensor data from the sensor;
detecting a location of the distal portion of the catheter within a luminal network of a patient;
determining a shape of the catheter within the luminal network of the patient;
modifying control parameters of the drive motor based on the detected location of the distal portion of the catheter and the determined shape of the catheter within the luminal network of the patient; and
driving the drive motor to articulate the distal portion of the catheter.

9. The system of claim 8, wherein the determined shape of the catheter is based on the received sensor data from the sensor.

10. The system of claim 8, wherein the application when executed by the processor:
compares the determined shape of the catheter to a pathway plan of the luminal network; and
modifies the determined shape of the catheter based on the comparison.

11. The system of claim 8, wherein modifying the control parameter adjusts one or more of a distance of pull of the at least one pull wire, a speed at which the at least one pull-wire is driven by the drive motor, or a force applied to the at least one pull wire by the drive motor to articulate the catheter.

12. The system of claim 8, wherein the drive motor is located in a handle connected to the catheter, the handle including one or more input devices for controlling the drive motor.

13. The system of claim 8, wherein the drive motor is operably connected to a robotic arm configured to advance the catheter within the luminal network of the patient.

14. The system of claim 8, wherein determining the shape of the catheter determines a location and angularity of at least one bend of the catheter.

15. The system of claim 8, wherein the detecting a location of a distal portion of the catheter, determination of the shape of the catheter, and modifying control parameters of the drive motor is repeated until a distal portion of the catheter arrives proximate a target.

16. The system of claim 8, wherein the sensor is one or more of a fiber, an electromagnetic (EM) sensor, an inertial measurement unit (IMU), or an ultrasonic sensor.

17. The system of claim 8, further comprising a second pull wire integrated into and extending the length of the catheter, the second pull wire operably connected to the drive motor and configured to articulate the catheter in a direction opposite the at least one pull wire.

18. The system of claim 17, further comprising a second drive motor operably connected to a second pair of pull wires, wherein the second pair of pull wires are integrated into and extend the length of the catheter and actuation of the second motor articulates the catheter in directions substantially orthogonal to a direction of articulation caused by the at least one pull wire and the second pull wire.

19. The system of claim 18, further comprising a third drive motor operably connected to the catheter and configured to rotate the catheter about its longitudinal axis.

20. The system of claim 8, wherein the sensor is an electromagnetic sensor configured to detect electromagnetic fields generated by a transmitter mat.
